(19) United States
(12) Patent Application Publication
(10) Pub. No.: US 2022/0378600 A1
(43) Pub. Date: Dec. 1, 2022

(54) UNIVERSAL SAFETY RESTRAINT CLIP FOR SINGLE PERSON DEPLOYABLE LIMB AND BODY RESTRAINTS

(71) Applicant: Ronald Gigliotti, Ashland, MA (US)

(72) Inventor: Ronald Gigliotti, Ashland, MA (US)

(21) Appl. No.: 17/827,634

(22) Filed: May 27, 2022

Related U.S. Application Data
(60) Provisional application No. 63/193,631, filed on May 27, 2021.

(51) Int. Cl. A61F 5/37

(52) U.S. Cl. CPC A61F 5/3769

(57) ABSTRACT

A safety restraint clip assembly attached to limb and body soft physical restraints. The safety restrain clip quickly and easily attaches and reattaches to standard railings less than 3 inches in diameter without removing the soft physical restraint on the patient.

### CROSS REFERENCE TO RELATED APPLICATION

This application claims the benefit of U.S. Provisional Patent Application No. 63/193,631 filed on May 27, 2022, the contents of which are incorporated herein by reference.

### FIELD OF THE INVENTION

The invention relates to universal safety restraint clip assembly for limb and body soft physical restraints.

### BACKGROUND OF INVENTION

During treatment at medical facilities, patients may often become physically violent due to psychological issues, confusion, disorientation, medical shock, or being under the influence of drugs or alcohol. To protect the patient from leaving the facility, moving without authorization within a facility, or self-injury, these patients must often be restrained. Additionally, patients must be restrained to keep their limbs fairly immobile in order for a certain treatment to be successful. Finally, to protect medical facility workers from injury, and the facility from damage, these patients must often be restrained.

Current medical facility restraints are inadequate to restrain violent patients in a timely fashion without physical injury. Current restraints are composed of long cloth theaters and padded wrist restraints that require three to four people to deploy onto the patient properly. During the length of time needed for deployment or removal, the patient or one of the workers may be injured.

Furthermore, current medical facility restraints cannot be universally deployed to restraint the patient to various types of ambulance stretchers, extrication equipment, and hospital stretchers. Current restraints are too bulky or specialized to use for universal restraint attachment.

In hectic and fast-paced facilities, three or four people may not be immediately available for restraint deployment. So, there is a significant need for a reliable physical restraint that can be deployed by one facility worker onto a patient. These restraints need to be lightweight, strong, and universally fit ambulance stretchers, extrication equipment, and hospital stretchers. With the universal fit, the restraint can remain attached to the patient, while moved from stretcher to stretcher, or other equipment.

### SUMMARY OF THE INVENTION

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The subject invention discloses a clip for a soft physical restraint, the clip comprising: a clip body comprising a main body with a first end and a second end, and two arms in substantially parallel alignment forming a substantially u-shaped opening within the clip; a pivotable clasp contained on the first end of the clip body on both arms, wherein the clasp is pivotable away from one arm to allow access into the u-shaped opening; wherein the substantially u-shaped opening is configured to fit over railing comprising a diameter of less than three inches between the two arms; a slot on the second end of the clip body, where the loop is configured to removably receive a substantially flat strap, wherein the flat strap is connected to the soft physical restraint that is configured to be releasably attachable to a patient's limb; and wherein the clip is configured to be moveable from railing to railing comprising a diameter of less than three inches, without removing the soft physical restraint attached to the patient's limb during the clip movement.

The subject invention discloses a clip for a soft body or limb restraint, the clip comprising: a clip body comprising a main body with a first end and a second end, and two arms in substantially parallel alignment forming an opening within the clip between the two arms; a pivotable clasp contained on the first end of the clip body on both arms, wherein the clasp is pivotable away from one arm to allow access into the opening; wherein the opening is configured to fit over railing comprising a diameter of less than three inches between the two arms; a slot on the second end of the clip body, where the loop is configured to removably receive a substantially flat strap, wherein the flat strap is connected to the soft body or limb restraint that is configured to be releasably attachable to a patient's body or limb; and wherein the clip is configured to be moveable from railing to railing comprising a diameter of less than three inches, without removing the soft body or limb restraint attached to the patient's body or limb during the clip movement.

In embodiments of the subject invention, the u-shaped opening may be configured to fit over the railing of rails selected from the group consisting of: ambulance stretchers, stair chairs, gurneys, extrication equipment, and hospital stretchers.

In further embodiments of the subject invention, the u-shaped opening may be four to five inches in height.

In other embodiments of the subject invention, the u-shaped opening may be configured to fit rails up to three inches in diameter.

In additional embodiments of the subject invention, the clip body may be four to eight inches in height.

In embodiments of the subject invention, the clip body may be three to five inches in width.

In other embodiments of the subject invention, the clip body may be 0.2 to one inch in thickness.

In further embodiments of the subject invention, the clip body may be composed of aluminum.

In embodiments of the subject invention, the strap may be comprise a material selected from the group consisting of: nylon webbing, leather, rubber, soft vinyl, and fabrics.

In other embodiments of the subject invention, the two arms of the clip body may each contain at least one external tactical gripping surface.

In embodiments of the subject invention, the term “substantially” is defined as at least close to (and can include) a given value or state, as understood by a person of ordinary skill in the art. In one embodiment, the term “substantially” refers to ranges within 10%, preferably within 5%, more preferably within 1%, and most preferably within 0.1% of the given value or state being specified.

In embodiments of the subject invention, the term “relatively” is defined as a comparison of a property, or the proportion of a property between two components.

### BRIEF DESCRIPTION OF THE DRAWINGS

Advantages of the present invention will be apparent from the following detailed description of embodiments, which description should be considered in conjunction with the accompanying drawings, in which:

FIG. 1 illustrates a top isometric view of one embodiment of the safety restraint clip and physical restraint strap assembly in the attached configuration.

FIG. 2 illustrates another top isometric view of the safety restraint clip and physical restraint strap assembly.

FIG. 3 illustrates a bottom isometric view of the safety restraint clip and physical restraint strap assembly.

FIG. 4 illustrates a top view of the safety restraint clip and physical restraint strap assembly.

FIG. 5 illustrates a left side view of the safety restraint clip and physical restraint strap assembly.

FIG. 6 illustrates a right side view of the safety restraint clip and physical restraint strap assembly.

FIG. 7 illustrates a top isometric view of the safety restraint clip and physical restraint strap assembly in the detached configuration.

FIG. 8 illustrates a top isometric view of the safety restraint clip in the closed position.

FIG. 9 illustrates another top isometric view of the safety restraint clip.

FIG. 10 illustrates a top view of the safety restraint clip.

FIG. 11 illustrates another top isometric view of the safety restraint clip.

FIG. 12 illustrates an exploded view of the safety restraint clip.

FIG. 13 illustrates a top view of the main body of the safety restraint clip.

FIG. 14 illustrates an isometric view of the main body of the safety restraint clip.

FIG. 15 illustrates a side view of the main body of the safety restraint clip.

FIG. 16 illustrates a front view of the main body of the safety restraint clip.

FIG. 17 illustrates an isometric view of the clasp of the safety restraint clip.

FIG. 18 illustrates a top view of the clasp of the safety restraint clip.

FIG. 19 illustrates a side view of the clasp of the safety restraint clip.

FIG. 20 illustrates an isometric view of the cam assembly of the physical restraint strap assembly.

FIG. 21 illustrates an exploded view of the cam assembly of the physical restraint strap assembly.

FIG. 22 illustrates an exploded view of the physical restraint strap assembly.

FIG. 23 illustrates an isometric view of the physical restraint strap assembly.

FIG. 24 illustrates another isometric view of the physical restraint strap assembly.

FIG. 25 illustrates a side view of a holster for safety restraint clip and physical restraint strap assembly.

FIG. 26 illustrates a front view of the holster.

FIG. 27 illustrates a rear view of the holster.

### DETAILED DESCRIPTION OF THE EMBODIMENTS

While several variations of the present invention have been illustrated in this application by way of example in particular embodiments, it is apparent that further embodiments could be developed within the spirit and scope of the present invention. However, it is to be expressly understood that such modifications and adaptations are within the spirit and scope of the present invention, and are inclusive, but not limited to the following appended claims as set forth.

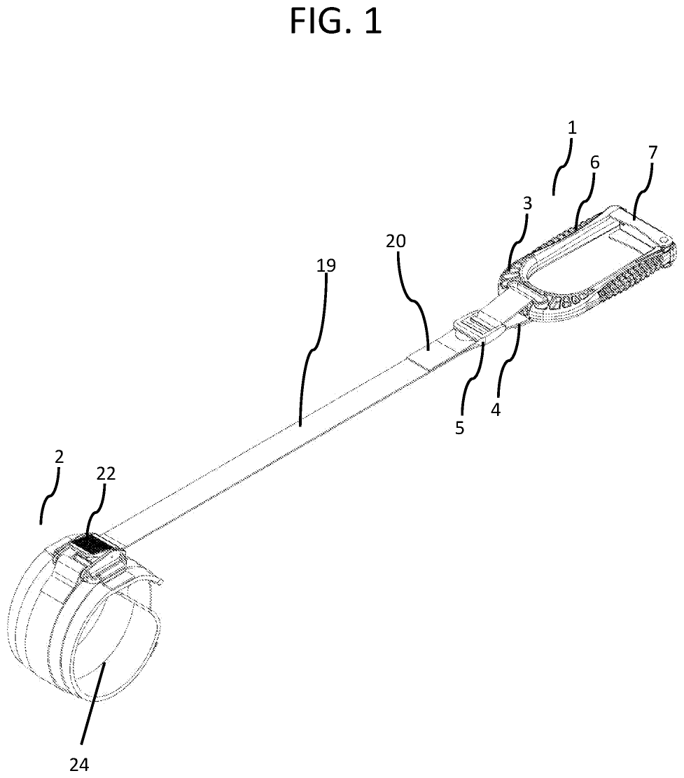

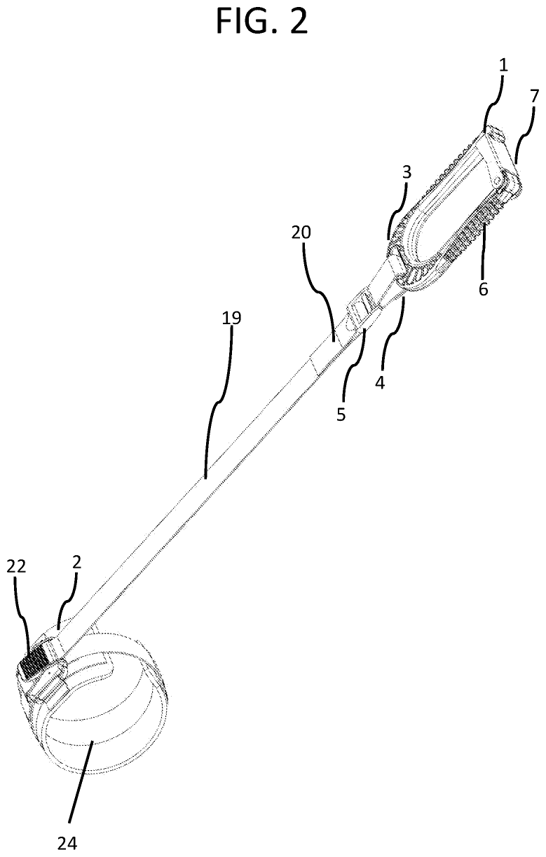

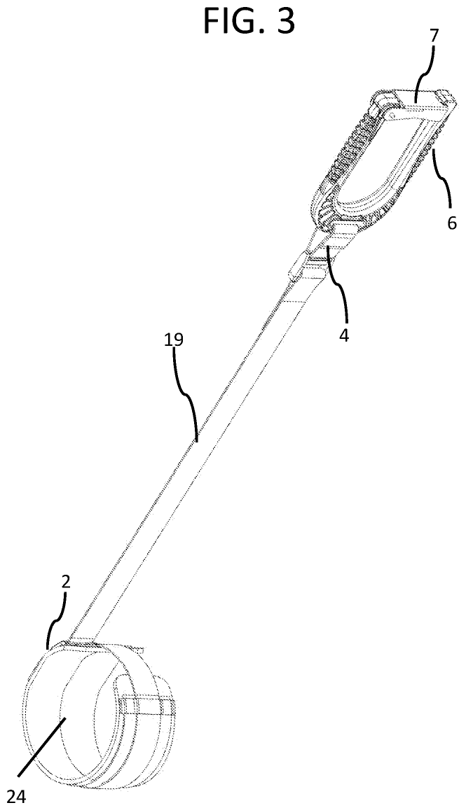

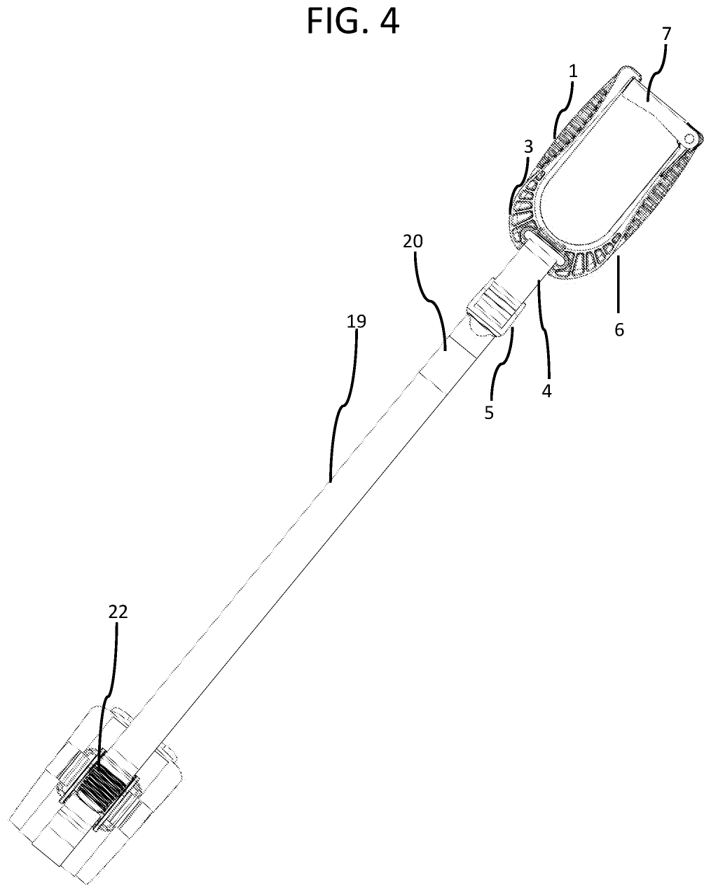

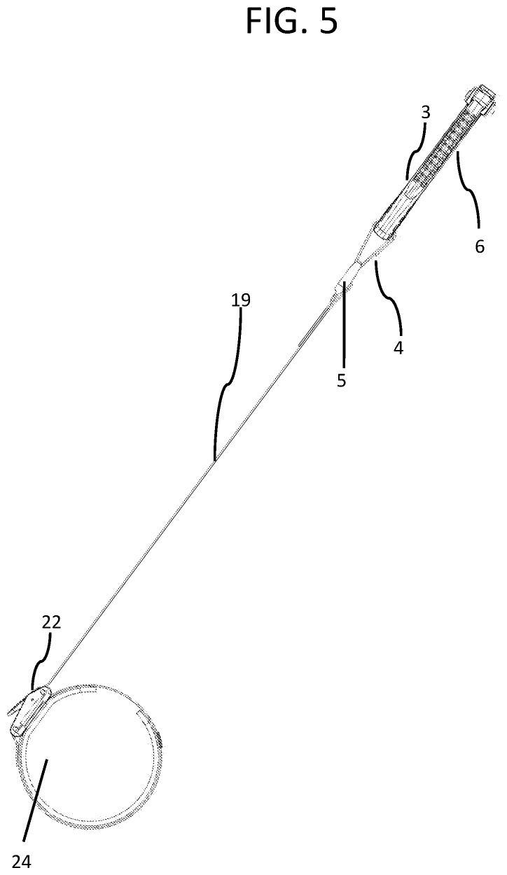

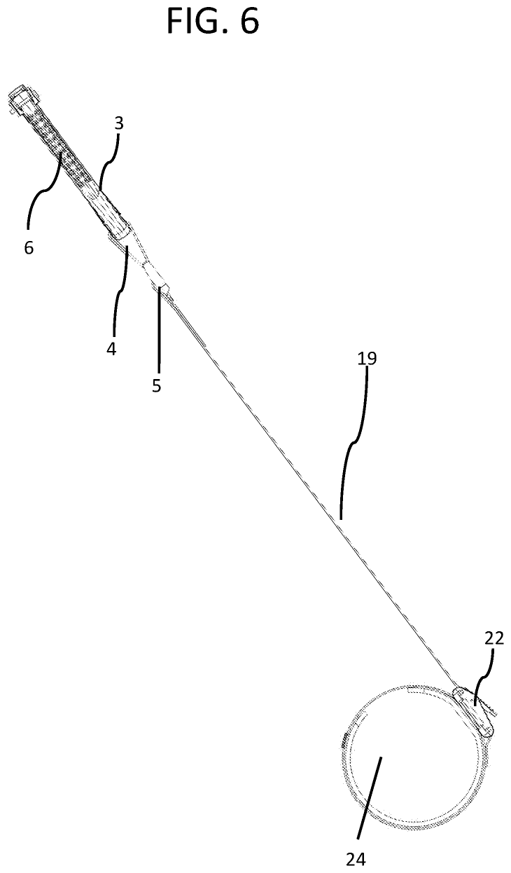

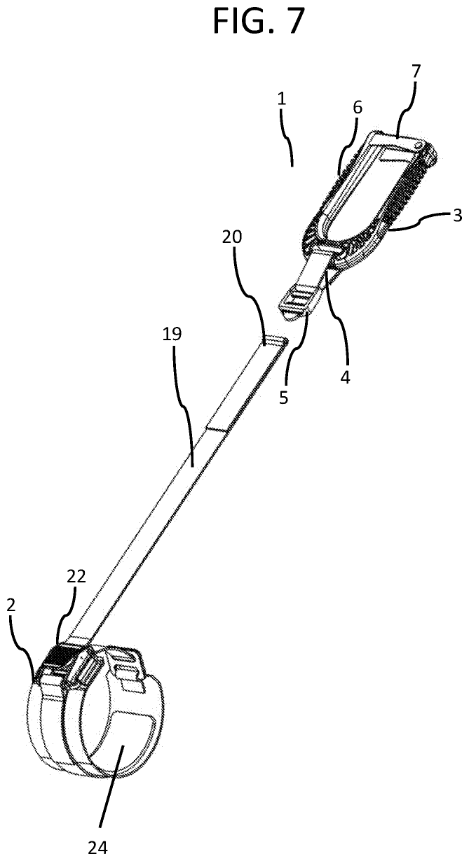

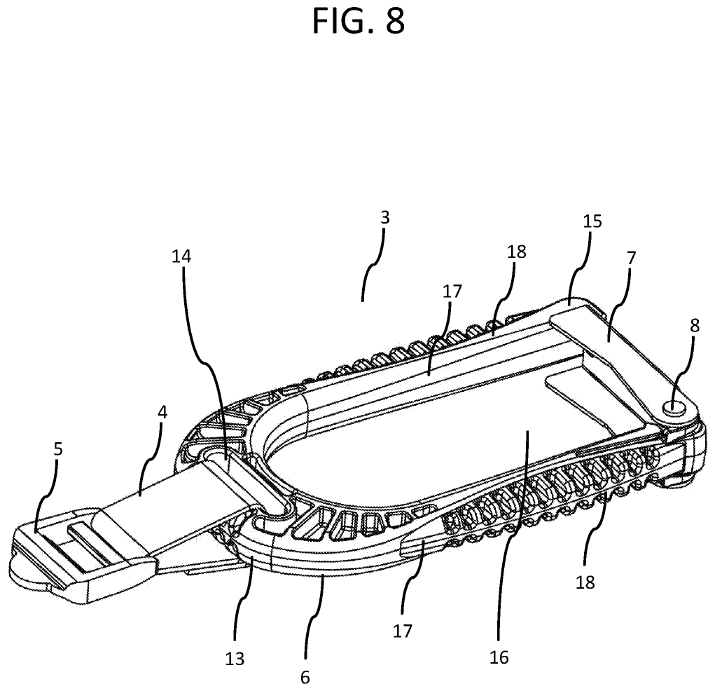

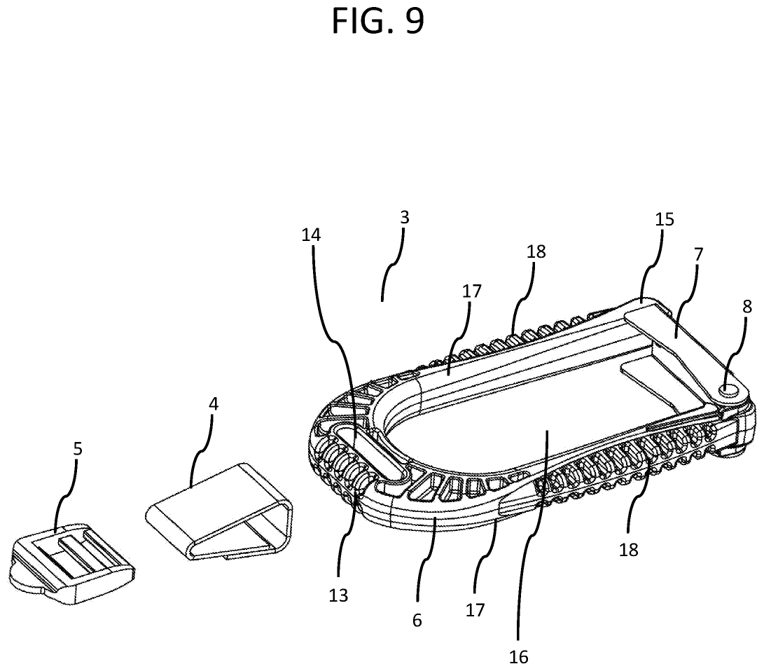

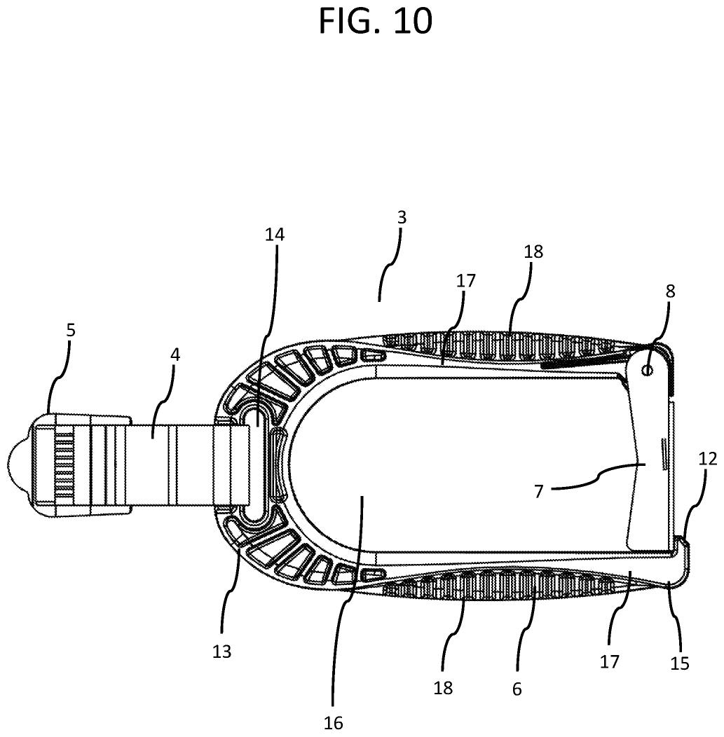

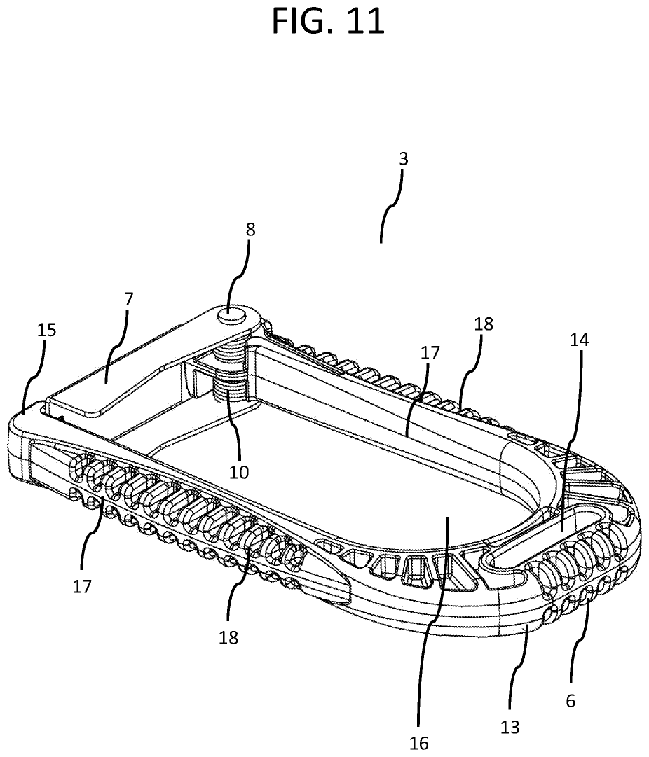

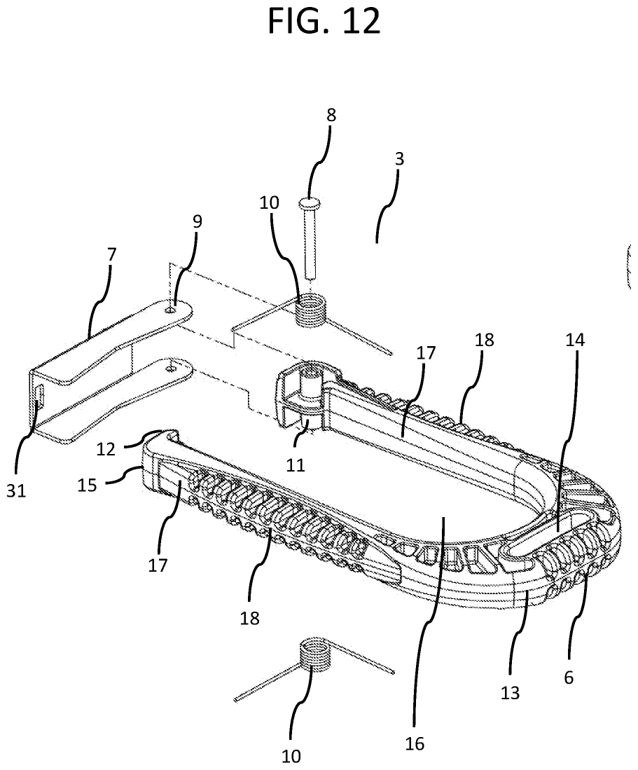

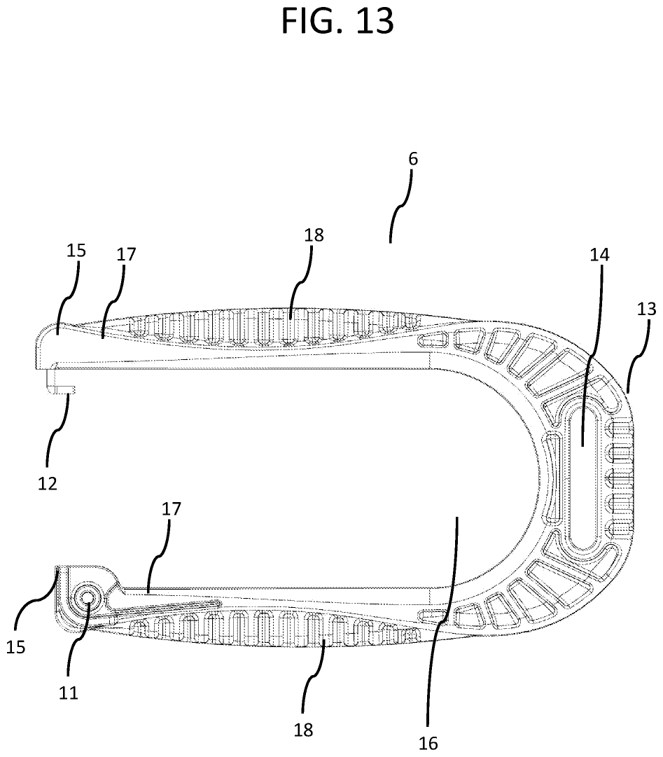

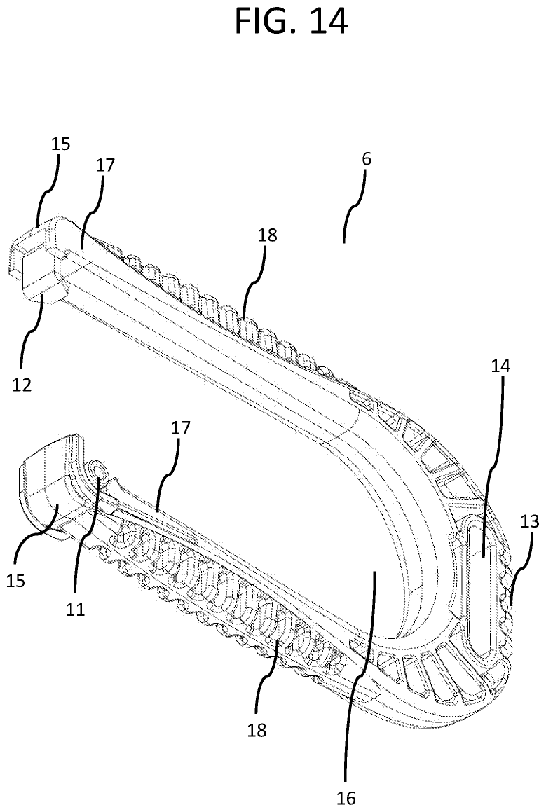

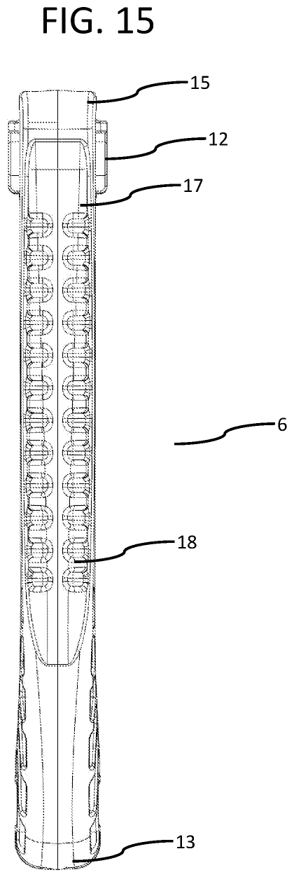

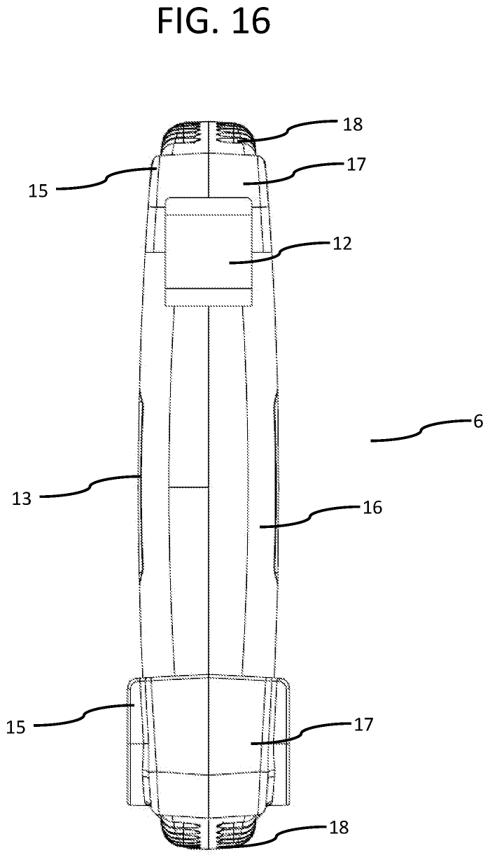

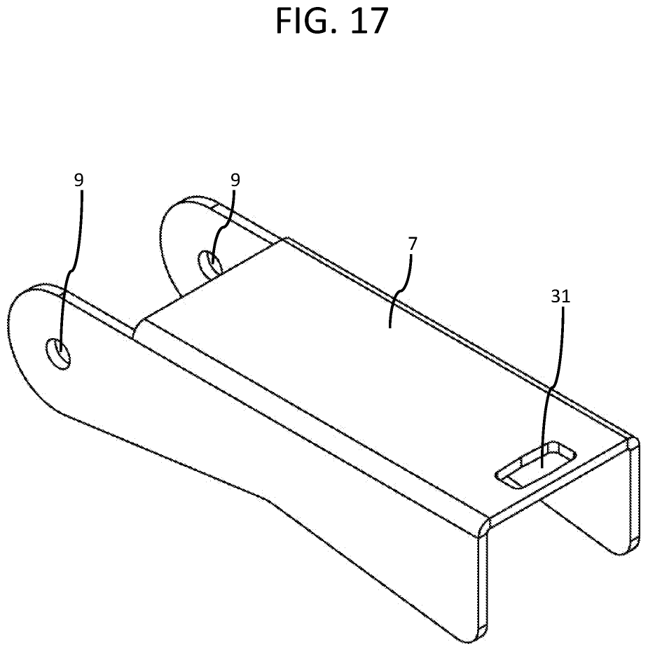

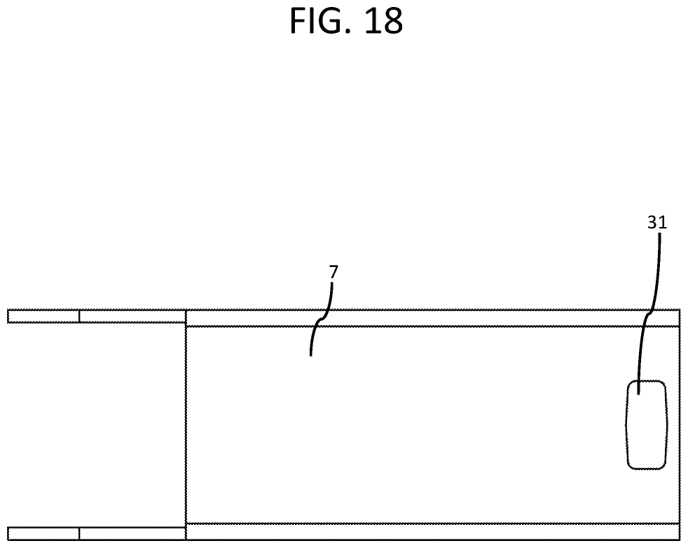

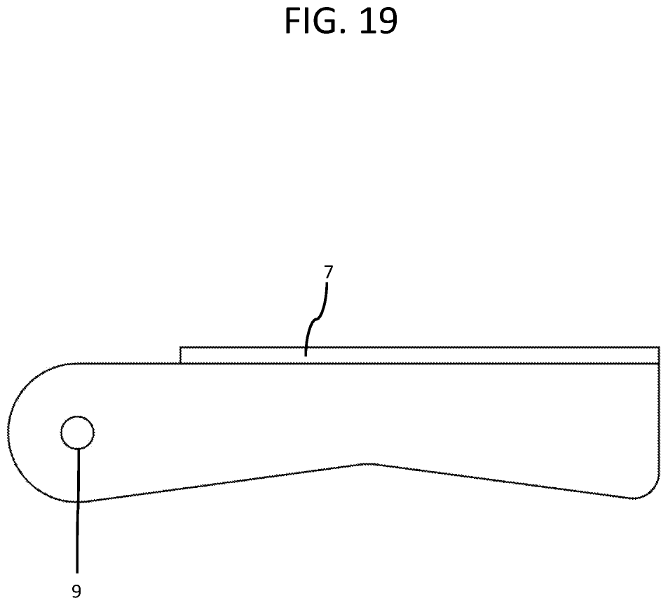

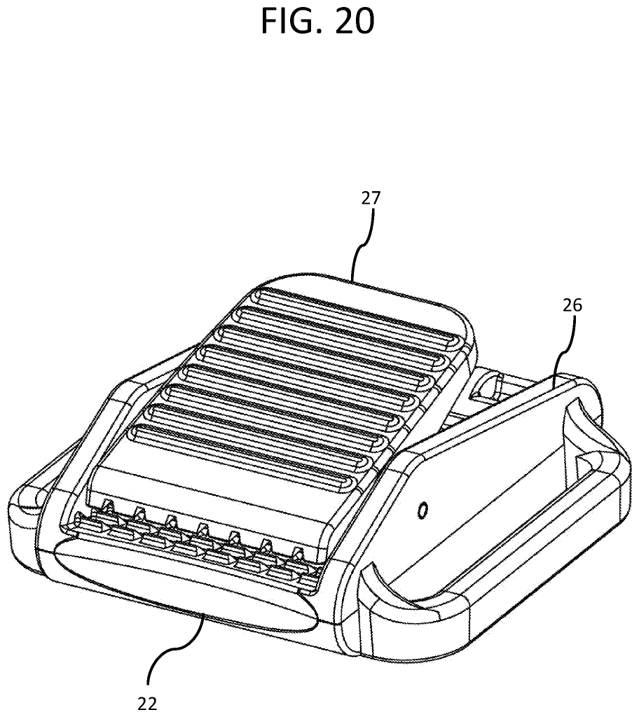

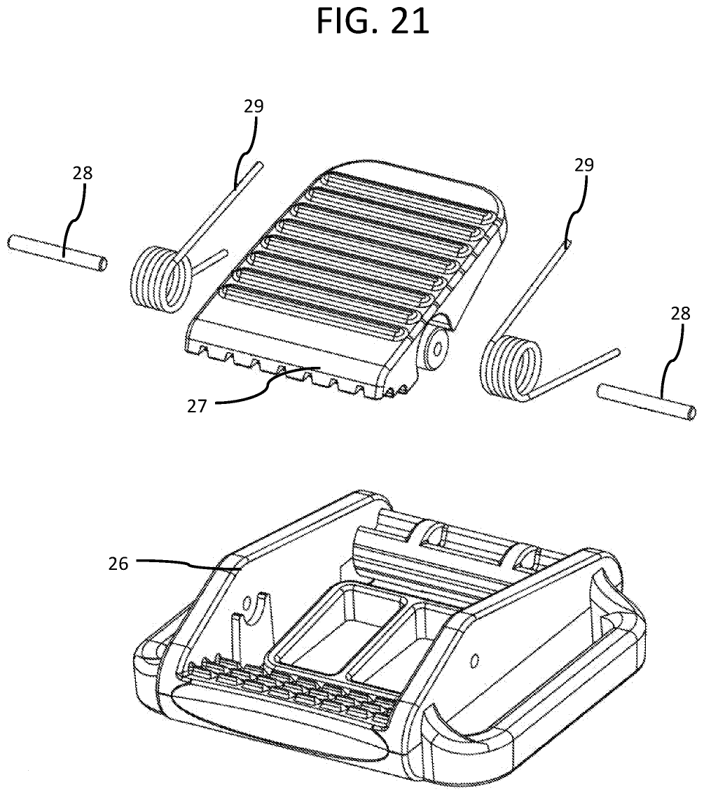

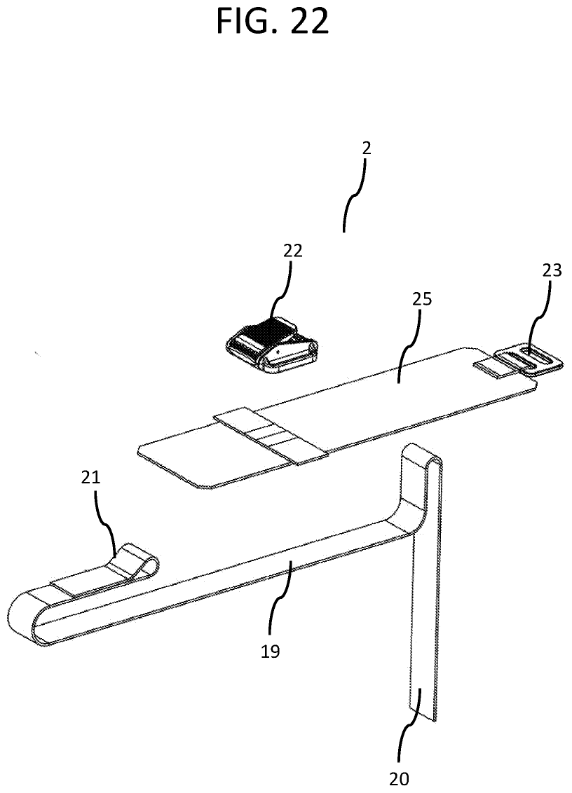

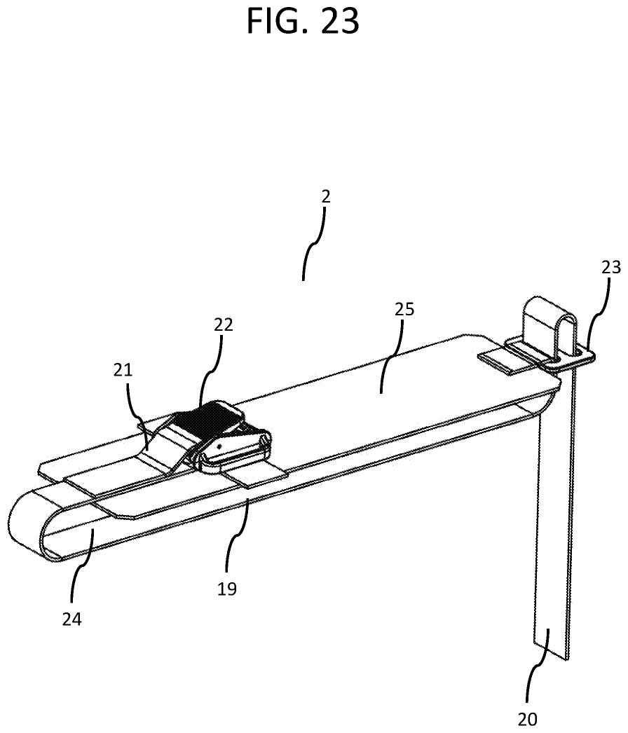

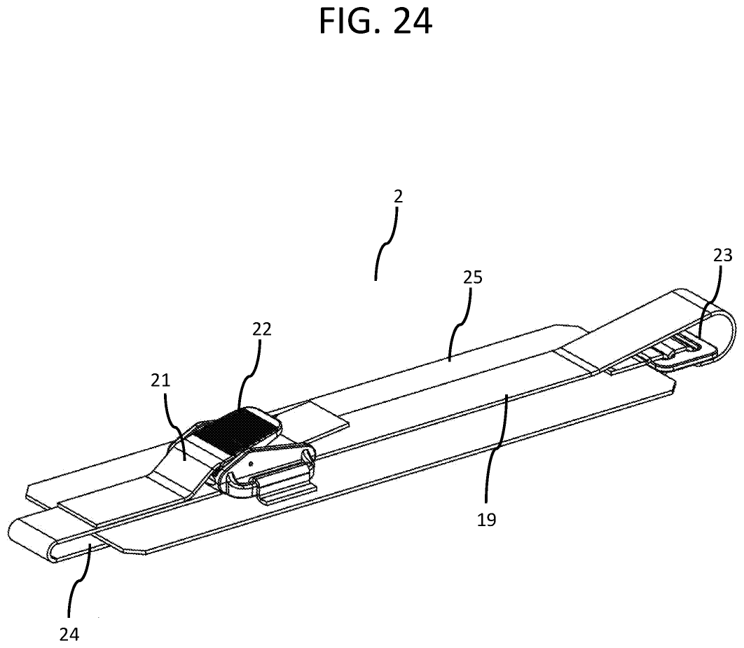

As illustrated in FIGS. 1-24, the subject invention, is a lightweight, strong, and compact safety restraint clip assembly 1 for universally usage with a single person limb and body physical strap assembly 2. The clip assembly 1 is designed to permit quick deployment of the physical strap assembly 2 on a patient.

As illustrated in FIGS. 1-16, the clip assembly 1 comprises a clip 3, a looped clip strap 4, and a clip buckle 5 for holding the looped clip strap 4.

The clip 3 comprises a main clip body 6 connected to a rotatable clasp 7 with a clip rivet 8 that passes through clasp 7 through a clasp holes 9, a clip torsion spring 10, and received in a clip body rivet hole 11. The main clip body contains a hook 12 that removably connects to a slot 31 on the clasp 7 for holding the clasp 7 in position on the main clip body 6 with the tension of the clip torsion spring 10. In embodiments of the subject invention, the clip buckle 5, the main clip body 6, the clasp 7, and the rivet 8 may be composed of any resilient, durable, lightweight materials known to those skilled in the art, including, but not limited to metals, such as steel or aluminum, metal alloys, plastics, or rubber materials.

In embodiments of the subject invention, the main clip body 6 may be four to eight inches in height, three to five inches in width, and 0.3 to 1 inches in thickness.

The first end 13 of the main clip body 6 contains a strap slot 14 for holding looped clip strap 4. In embodiments of the subject invention, the clip strap 4 may be composed of any strap materials known to those skilled in the art, including, but not limited to: nylon webbing, leather, rubber, soft vinyl, or other fabric straps.

The second end 15 of the main clip body 6 contains a substantially U-shaped opening 16 formed by two clip arms 17, which are substantially aligned and parallel with each other. The U-shaped opening 16 is configured to receive various rail diameter sizes on ambulance stretchers, stair chairs, gurneys, extrication equipment, and hospital stretchers. In embodiments of the subject invention, the U-shaped opening 16 may be four to five inches in height, three to four inches in width and will fit rails up to three inches in diameter. The main clip body 6 contains external tactical grips 18 on the external side of each clip arm 17.

As illustrated in FIGS. 1-17 and 20-24, physical strap assembly 2 is for connection to a patient's limb or body. The clip assembly 1 is removably attached to the physical strap assembly 2 with a main restraint strap 19 looped on one end 20 through the clip buckle 5. In embodiments of the subject invention, the main restraint strap 19 may be composed of any strap materials known to those skilled in the art, including, but not limited to: nylon webbing, leather, rubber, soft vinyl, or other fabric straps.

The other end 21 of the main restraint strap 19 is looped through one end of a cam assembly 22, a slide 23, and forms a loop 24 to connect to the other end of the cam assembly. The loop 24 is placed around a patient's limp or body, and has an adjustable diameter. The internal surface of the loop 24 may contain a pad 25 for additional patient comfort and restraint stability. In embodiments of the subject invention, the pad 25 may be composed of any strap materials known to those skilled in the art, including, but not limited to: nylon webbing, leather, rubber, soft vinyl, or other fabrics.

The cam assembly 22 is composed of a cam body 26 attach to a cam lever 27 with a pin 28 and a cam torsion spring 29. The cam body 26 and the cam level 27 contain the main restraint strap 19 with tension of the cam torsion spring 29. By lifting the cam level 27, the main restraint strap 19 can be adjusted to remove the strap 19 from the cam assembly 22 or adjust the diameter of the loop 24. In embodiments of the subject invention, the cam assembly 22, the slide 23, the cam body 26, the cam lever 27, and the pin 28 may be composed of any resilient, durable, lightweight materials known to those skilled in the art, including, but not limited to metals, such as steel or aluminum, metal alloys, plastics, or rubber materials.

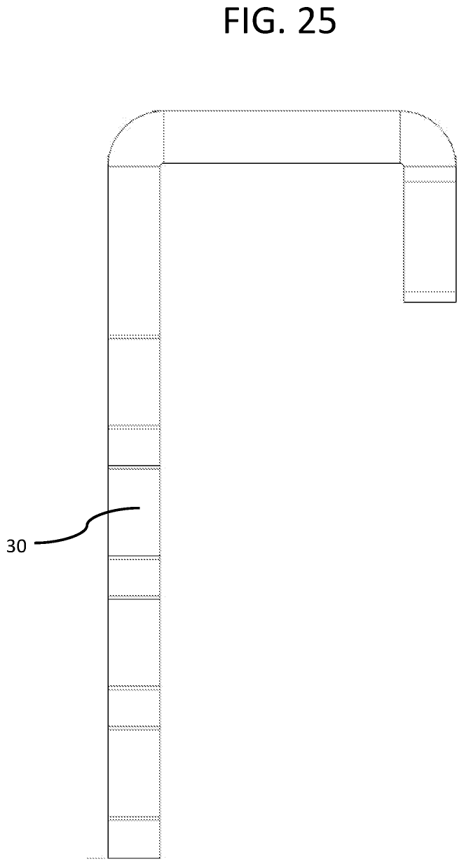

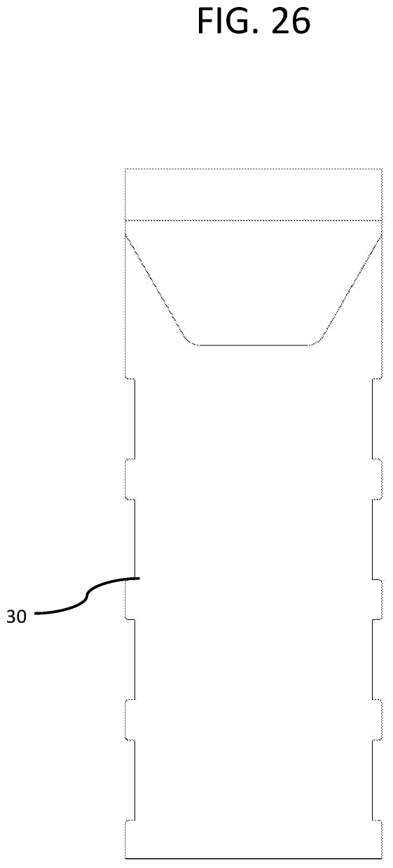

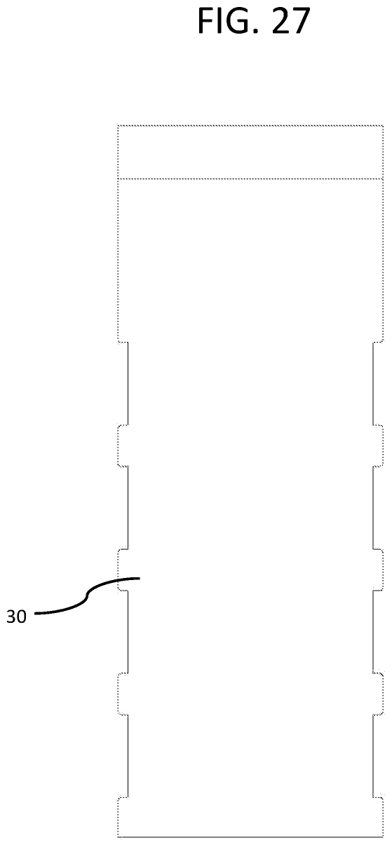

As illustrated in FIGS. 25-27, when not in use, the clip assembly 1 and the physical strap assembly 2 may be folded and contained on a holster 30. In additional embodiments of the subject invention, the clip assembly 1 and the physical strap assembly 2 may fit within a standard holster for quick retrieval and deployment. In embodiments of the subject invention, the holster 30 may be composed of any resilient, durable, lightweight materials known to those skilled in the art, including, but not limited to metals, such as steel or aluminum, metal alloys, plastics, leather, or rubber materials.

When deployed, the clip assembly 1 and physical strap assembly 2 are unfolded. The main restraint strap 19 may be loosened so that loop 24 slides over a patient's limb or body. The loop 24 has an adjustable diameter. In other embodiments, the loop 24 is broken by detaching the main restraint strap 19 from the cam assembly 24 or the slide 23. By lifting the cam level 27, the main restraint strap 19 can be adjusted to remove the strap 19 from the cam assembly 22 or adjust the diameter of the loop 24. The loop 24 is then reformed around the patient's limb or body by re-looping the main restraint strap 19 through the cam assembly 24 or the slide 23.

The length physical main restraint strap 19 can be adjusted as needed with the cam assembly 22 to secure the patient and for the patient's comfort.

The slot 31 on clasp 7 on the main clip body 6 is unhooked from hook 12, and the clasp 7 is rotated into an open position about the clasp holes 9. The U-shaped opening 16 is placed over a rail on an ambulance stretches, stair chair, gurney, extrication equipment, or a hospital stretcher, so that the rail is contained between the two clip arms 17 within the U-shaped opening 16. The clasp 7 is rotated into a closed position about the clasp holes 9 and the slot 31 on clasp 7 on the main clip body 6 are hooked onto hook 12, securing the patient to the desired railing.

The clip assembly 1 can be moved from railing to railing on stretcher to stretcher, or other equipment by repeating the clasp 7 opening and closing process, without removing the physical strap assembly 2 from the patient. The slot 31 on clasp 7 on the main clip body 6 is unhooked from hook 12, and the clasp 7 is rotated into an open position about the clasp holes 9. The U-shaped opening 16 is removed from the first rail and placed over a second rail, so that the second rail is contained between the two clip arms 17 within the U-shaped opening 16. The clasp 7 is rotated into a closed position about the clasp holes 9 and the slot 31 on clasp 7 on the main clip body 6 are hooked onto hook 12, securing the patient to the desired second rail.

The devices, systems, and methods disclosed herein are not to be limited in scope to the specific embodiments described herein. Indeed, various modifications of the devices, systems, and methods in addition to those described will become apparent to those of skill in the art from the foregoing description.

1. A clip for a soft physical restraint, the clip comprising:
a clip body comprising a main body with a first end and a second end, and two arms in substantially parallel alignment forming a substantially u-shaped opening within the clip;
a pivotable clasp contained on the first end of the clip body on both arms, wherein the clasp is pivotable away from one arm to allow access into the u-shaped opening;
wherein the substantially u-shaped opening is configured to fit over railing comprising a diameter of less than three inches between the two arms;
a slot on the second end of the clip body, where the loop is configured to removably receive a substantially flat strap, wherein the flat strap is connected to the soft physical restraint that is configured to be releasably attachable to a patient's limb; and
wherein the clip is configured to be moveable from railing to railing comprising a diameter of less than three inches, without removing the soft physical restraint attached to the patient's limb during the clip movement.

2. The clip of claim 1, wherein the u-shaped opening is configured to fit over the railing of rails selected from the group consisting of: ambulance stretchers, stair chairs, gurneys, extrication equipment, and hospital stretchers.

3. The clip of claim 1, wherein the u-shaped opening is four to five inches in height.

4. The clip of claim 1, wherein the u-shaped opening is configured to fit rails up to three inches in diameter.

5. The clip of claim 1, wherein the clip body is four to eight inches in height.

6. The clip of claim 1, wherein the clip body is three to five inches in width.

7. The clip of claim 1, wherein the clip body is 0.2 to one inch in thickness.

8. The clip of claim 1, wherein the clip body is composed of aluminum.

9. The clip of claim 1, wherein the strap comprises a material selected from the group consisting of: nylon webbing, leather, rubber, soft vinyl, and fabrics.

10. The clip of claim 1, wherein the two arms of the clip body each contain at least one external tactical gripping surface.
